(19) United States
(12) Patent Application Publication
(10) Pub. No.: US 2018/0333528 A1
(43) Pub. Date: Nov. 22, 2018

(54) Method and Devices for Providing a Solution for the Treatment of Blood

(71) Applicant: Fresenius Medical Care Deutschland GmbH, Bad Homburg (DE)

(72) Inventor: Jürgen Klewinghaus, Oberursel (DE)

(21) Appl. No.: 15/530,262

(22) PCT Filed: Jun. 15, 2015
(86) PCT No.: PCT/EP2015/063334

§ 371 Date: Aug. 1, 2018

(30) Foreign Application Priority Data
Jun. 16, 2014 (DE) 10 2014 108 444.6

(51) Int. Cl. A61M 1/34; A61M 1/16; A61M 1/36

(52) U.S. Cl. CPC A61M 1/3462; A61M 1/1656; A61M 1/1603; A61M 1/3627; A61M 1/3413; A61M 2205/36; A61M 2205/3393; A61M 2205/50; A61M 1/3624; A61M 2205/3306; A61M 2205/3379

(57) ABSTRACT

The present invention relates to a blood treatment apparatus: at least one first source for a saline solution, a fresh dialysate, or a substituate as a first fluid; at least one first conduit portion which connects to the first source; at least one second source of a second fluid in the form of a concentrate or a solution; at least one second conduit portion which connects to the second source; at least one third conduit portion, which is in fluid communication with a section of the extracorporeal blood circuit to which both the first conduit portion and the second conduit portion are fluidly connected such that both the first source and the second source are in fluid communication with the third conduit portion; a first fluid conveying device, arranged to convey a fluid present inside the third conduit portion. The invention further relates to a corresponding method.

The present invention relates to a method according to claim 1 for providing a solution for the blood treatment. In addition, it relates to a control unit according to claim 10 and a blood treatment apparatus according to claim 11. Furthermore, the present invention relates to a digital storage medium according to claim 20, a computer program product according to claim 21 and a computer program according to claim 22.

In practice, an active agent or substance (hereinafter denoting in short medicament, drug and/or pharmacologically active substance) is added in some extracorporeal blood treatment sessions to a thereby utilized blood circuit.

The addition of the solution is carried out in practice using a container, e.g. a solution bag (or in short: bag), in which the agent or substance is present in a solution. The solution may have a variously high concentration. The volume of the solution for reaching a determined concentration of the agent in the blood obviously depends on the concentration which the agent has in the solution. Herby, both high-concentration and low-concentration solutions have each their advantages and disadvantages.

An advantage of solution bags with medicament in high concentrations is that a utilized bag lasts comparatively long and the bag-change intervals can be long, which saves time for changing the bag, and which requires very little storage space for such bags. A disadvantage of using bags with high concentration is that highly concentrated solutions tend to rather mix with the patient's blood in an uneven way, e.g. clot formation.

An advantage of using bags with low concentration is that their solution does not rather lead to clot formation and at the same time it mixes with the patient's blood more evenly. A disadvantage of bags with low concentration is that a used solution bag holds comparatively short and bag-change intervals are thus brief costing therefore time to change the bag. In addition, such bags require a relatively large storage space causing increased costs for procurement and disposal.

It is an object of the present invention to specify a further method and devices for providing a solution for the blood treatment.

Further, a blood treatment apparatus by means of which the method according to the present invention is feasible as well as a control unit provided for executing the method according to the present invention, a suitable digital storage medium, a suitable computer program product and a suitable computer program are specified.

The object according to the present invention is solved by means of a method with the features of claim 1. It is further accomplished by means of a control unit with the features of claim 10 as well as a blood treatment apparatus with the features of claim 11. The object according to the present invention is further accomplished by means of a digital storage medium, a computer program product as well as a computer program according to the claims 20, 21 and 22.

All advantages achievable by means of the method according to the present invention may in certain embodiments according to the present invention be achieved in an undiminished manner also by the apparatuses according to the present invention.

The method according to the present invention relates to providing a solution for a blood treatment of a patient which is carried out by means of a blood treatment apparatus and by using an extracorporeal blood circuit and/or a blood cassette. The method encompasses at least providing a blood treatment apparatus which for the purpose of the treatment of the patient's blood comprises, is connectable or connected, in fluid and or signal connection with devices and apparatuses that are mentioned below.

Thus, the blood treatment apparatus comprises or is connected with at least one first source with or for a (preferably physiological) saline (e.g. a 0.9%-NaCl solution), a fresh dialysate or a substituate (i.e. a substituate fluid) or another suitable solution or fluid as a first fluid. It further comprises or is connected with one first conduit which connects downstream to the first source and/or is in fluid communication therewith. To facilitate matters, The first fluid is indicated in the following as fresh dialysate of substituate, which should not present any restriction or limitation. The first fluid may as indicated or explained above be, e.g., a saline solution. The below-mentioned elaboration about the fresh dialysate or substituate, applies undiminished also for saline solution and the like, provided the skilled in the art has no objection to this assumption.

The blood treatment apparatus comprises further at least one source for an agent or substance which is available as a second fluid in the second source as concentrate or a solution or is connected thereto.

The blood treatment apparatus comprises further at least one second conduit which connects downstream to the second source and/or which is in fluid communication therewith.

The blood treatment apparatus further comprises at least one so-called joint or common conduit. This joint or common conduit is in fluid communication with, possibly, a further section of the extracorporeal blood circuit and/or with, possibly, a further section of the blood cassette, preferably with an arterial or a venous conduit section. It is denoted as a joint or common conduit because both the first and second fluid may be present in it and flow jointly or commonly, i.e. at the same time. For this purpose, the first conduit and the second conduit open or merge into the joint or common conduit such that both the first source and the second source are in fluid communication with the joint or common conduit and/or the first fluid flowing from the first source and the second fluid flowing from the second source meet in the joint or common conduit due to the design of the latter forming a combined or mixed fluid.

The blood treatment apparatus further comprises at least one first conveying device for conveying a fluid. The first conveying device is arranged to convey the fluid present inside the joint or common conduit, preferably in or within the extracorporeal blood circuit and/or blood cassette.

The method according to the present invention further encompasses operating at least the first conveying device such that a combination of the first fluid and the second fluid is conveyed into the joint or common conduit and preferably into an arterial or venous conduit section of the extracorporeal blood circuit or blood cassette.

The control unit according to the present invention is suitable and provided and/or arranged and/or configured for executing the method according to the present invention.

The blood treatment apparatus according to the present invention comprises an extracorporeal blood circuit and/or a blood cassette or is connected thereto. The blood treatment apparatus further comprises or is connected with at least one first source for a fresh dialysate or a substituate as a first fluid; at least one first conduit which connects downstream to the first source at least one second source for an agent or substance which is present as a second fluid in the second source as a concentrate or a solution; at least one second conduit which connects downstream to the second source; at least one joint or common conduit which is in fluid communication with a section of the extracorporeal blood circuit and/or a section of the blood cassette, and into which both the first conduit and the second conduit flow or pass or merge such that both the first source and the second source are in fluid communication with the joint conduit and/or wherein the joint or common conduit is arranged such that the first fluid flowing from the first source and the second fluid flowing from the second source meet inside of it forming a combined or mixed fluid; and a first fluid conveying device arranged to convey a fluid present inside the joint or common conduit.

The blood treatment apparatus according to the present invention is provided and/or arranged and/or equipped for executing the method according to the present invention.

The control unit according to the present invention is suitable and provided and/or arranged and/or configured for executing the method according to the present invention in interaction with each device required for this purpose, as described in the following by way of example.

A digital, particularly a non-volatile storage medium according to the present invention, particularly as a machine-readable data storage device, particularly as a disk, CD, EPROM or DVD, with electrically readable control signals may interact with a programmable computer system such that the mechanical steps of a method according to the present invention are prompted.

In doing so, all or some of the mechanically executed steps of the method according to the present invention may be prompted.

A computer program product according to the present invention comprises a program code that is volatile or saved on a machine-readable medium for prompting the mechanical steps of the method according to the present invention when the computer program product runs on a computer. According to the present invention a computer program product can be understood as, for example, a computer program which is stored on a storage device, an embedded system as a comprehensive system with a computer program (e.g. an electronic device with a computer program), a network of computer-implemented computer programs (e.g. a client-server system, a cloud computing system, etc.), or a computer on which a computer product is loaded, executed, saved or developed.

The term machine-readable medium as used herein denotes in certain embodiments of the present invention a medium containing data or information which is interpretable by software and/or hardware. The medium may be a data medium, like a disk, a CD, DVD, a USB stick, a flashcard, an SD card or the like.

A computer program according to the present invention comprises a program code for prompting the mechanical steps of a method according to the present invention when the computer program runs on a computer. A computer program according to the present invention can be understood as, for example, a physical, ready-for-distribution software product which comprises a computer program.

It also applies for the computer program product according to the present invention and the computer program according to the present invention that all or some of the mechanically executed steps of the method according to the present invention are prompted.

Embodiments according to the present invention may comprise some or all of the following features in arbitrary combination, provided this is not realized by the skilled person to be technically impossible. Advantageous developments of the present invention are each also subject of the dependent claims.

In all of the following embodiments, the use of the expression may be or may have and so on, is to be understood synonymously with preferably is or preferably has, respectively, and so on, and is intended to illustrate exemplary embodiments according to the present invention.

Whenever a numerical word is mentioned herein, the skilled person understands this as an indication of a numerically lower limit. Therefore, the skilled person, implicitly reads at all times, provided it does not lead to any discernible contradiction for the skilled person, for example in the indication “one” “at least one”. This understanding is also encompassed by the present invention as well as the interpretation that, for example, “one” can alternatively be meant as “exactly one”, as long as this is technically possible in the view of the skilled person. Both of which are encompassed by the present invention and apply to all used numerical words herein.

In some particular exemplary embodiments according to the present invention, only the first and the second conduit open or merge into the joint or common conduit, however no third or further conduit.

In certain exemplary embodiments according to the present invention, the joint or common conduit, however when used as intended, does not conduct any blood. The joint or common conduit is disposed at a corresponding place. Thereby, the joint or common conduit may for its part open or merge into a blood conducting further conduit.

In some exemplary embodiments according to the present invention, the joint or common conduit and/or the first conveying device is/are arranged such that no fluid present in the joint or common conduit may be or may get conveyed by means of the blood pump.

In some particular exemplary embodiments according to the present invention, merely the first and the second fluid form or build the combined or mixed fluid which is present in the joint or common conduit. Other fluids are not involved, in these embodiments, in the composition of the mixed or combined fluid. Other sources as the first source and the second source, in particular upstream of first conveying device, are not in fluid communication with the joint or common conduit.

In some exemplary embodiments according to the present invention, the joint or common conduit is not connected to a water source or blood source or fed by such sources.

In certain exemplary embodiments according to the present invention, the fresh dialysate or substituate is available in the first source as ready-to-use. In particular, no mixing with water is required in this process for producing a ready-to-use fresh dialysate or a ready-to-use substituate out of the contents of the first source.

In some exemplary embodiments according to the present invention, the first and/or the second fluid are present in the first or the second source as liquid, however not as powder and/or in a dry form.

In some particular exemplary embodiments according to the present invention, the first and/or the second source are a bag.

In some exemplary embodiments according to the present invention, there is no mixing of a fresh dialysate, a substituate or the mixed or combined fluid which is present in the joint or common conduit in one or several tanks.

In certain exemplary embodiments according to the present invention, one or two conveying devices, which may convey the content of the joint or common conduit, are arranged upstream of a point at which the joint or common conduit opens up or merges into a blood conducting section of the extracorporeal blood circuit or of the blood cassette.

In certain exemplary embodiments according to the present invention, the blood treatment apparatus comprises or is connected with at least one second conveying device. The method further encompasses conveying the second fluid from the second source into the second conduit and/or into the joint or common conduit by means of the second conveying device.

In certain exemplary embodiments of the present invention, the second conveying device is not a conveying device which exclusively or partially conveys the second fluid by means of gravity or capillary effect or action.

In some exemplary embodiments according to the present invention, the method encompasses conveying the first fluid from the first source into the first conduit and conveying the second fluid from the second source into the second conduit in a determined or adjustable volume relation or flow relation, in particular determined and/or adjusted by the user, with reference to the first and second fluid.

In certain exemplary embodiments according to the present invention, the method encompasses monitoring a volume conveyed by means of the first conveying device and/or the second conveying device into the joint conduit or monitoring a respective feed of the first conveying device and/or the second conveying device into the joint conduit, each by means of a first and/or second monitoring device.

In some exemplary embodiments according to the present invention, the method encompasses determining, by means of the first and/or the second monitoring device, a distance by which a section of the second conveying device, e.g. a stamp, is moved. The method further comprises comparing, by means of a comparing device, the detected distance with reference data.

In specific exemplary embodiments according to the present invention, the method encompasses counting the drops of the second fluid by means of a drop counter. The method further encompasses comparing, by means of a comparing device, the detected number of drops with reference data.

The reference data may herein be understood as data areas. They may be lodged as tables, stored electronically, etc.

Signs or warnings are issued in case of deviations of the calculated number of drops to the reference data. A mixing relation between the first and the second fluid may be, alternatively or additionally, automatically adjusted depending on or as a reaction to the detected deviation.

In some particular exemplary embodiments according to the present invention, the method encompasses detecting a weight change of the first source by means of at least one scale. The feed rate or the volume conveyed by the first conveying device the can be concluded based on the detected change of weight. This may take place in a comparing, adjusting or monitoring manner. The result of which may be used for corrective intervention the controlling of the first conveying device, which may hereby be a regulation. The result of comparing, adjusting or monitoring may be conveyed or issued to the user as information or alarm.

When concluding from the detected weight changes to the feed rate of the first conveying device, this may exemplarily be calculated, taken from a table or approximately determined or estimated. Hereby, additional information such as the knowledge of a portion of the second fluid coming from the second source, at or in the combination of the first and second fluid conveyed by means of the first conveying device may be considered.

In some exemplary embodiments according to the present invention, the second source comprises—preferably exclusively, substantially or mainly—citrate solution, calcium solution or heparin solution. In certain exemplary embodiments according to the present invention, the second source comprises no electrolytes or comprises only certain electrolytes (in any combination). These include, within the context of the present invention, bicarbonate, chloride, copper, glucose, iron, magnesium, phosphate, sodium, and zinc.

A citrate solution comprises citrate in some embodiments according to the present invention, as described in US 2006/0037910 A1 and in particular in its paragraphs [0040] to [0042]. The relevant disclosure is made the subject of also the present application by reference.

In certain exemplary embodiments according to the present invention, the blood treatment device is a hemodialysis apparatus, haemofiltration apparatus or haemodiafiltration apparatus.

In some exemplary embodiments according to the present invention, the blood treatment apparatus comprises a first monitoring device for monitoring the pumping function of the first conveying device.

In certain exemplary embodiments according to the present invention, the blood treatment device comprises a second monitoring device for monitoring the pumping function of the second conveying device.

In some exemplary embodiments according to the present invention, the second conveying device is or comprises a syringe pump with a stamp. The second monitoring device is configured to monitor the pumping function of the second conveying device. Therefore, the distance by which the stamp of the second conveying device is moved, is detected. The result of the detection is compared with reference data by means of a comparing device.

In certain exemplary embodiments according to the present invention, the first and/or the second monitoring device are configured to monitor the pumping function of the first and/or the second conveying device. For this purpose, they monitor the function of each conveying device by counting the drops of the first and/or second fluid. The respective conveying device is monitored through comparing the detected number of drops with reference data.

In some exemplary embodiments according to the present invention, the blood treatment apparatus comprises one control or regulating unit, preferably according to the present invention. The control or regulating unit may be programmed and/or configured for executing the method according to the present invention in interaction with further devices, in particular a blood treatment apparatus.

The extracorporeal blood circuit is in certain embodiments according to the present invention a tube set. In any case, the extracorporeal blood circuit is provided for extracorporeally conducting blood of a patient, e.g., during hemodialysis, hemofiltration, hemodiafiltration or the like.

In some embodiments according to the present invention, the extracorporeal blood circuit is at least in sections embodied as an integral and, where appropriate, permanent part of the functional device, in others it is not. A freely movable tube section of the extracorporeal blood circuit may therefore continue in one piece or integrally on or in the functional device, e.g., a blood cassette, and vice versa.

A blood cassette is in certain embodiments according to the present invention a device which is used in a blood treatment. Examples of blood cassettes include disposables, one-time blood cassettes.

Exemplary embodiments of a blood cassette are in particular disclosed in the application of the Applicant with the publication number DE 10 2009 018 664 A1 having the title Externe Funktionseinrichtung, Blutbehandlungsvorrichtung zum Aufnehmen einer erfindungsgemäßen externen Funktionseinrichtung, sowie Verfahren, which was submitted to the German Patent and Trademark Office on 23 Apr. 2009, and in the application of the Applicant with the publication number DE 10 2009 024 468 A1 of the same title, which was submitted to the German Patent and Trademark Office on 10 Jun. 2009. The respective disclosures are herewith fully incorporated by way of reference.

The arterial conduit section of the extracorporeal blood circuit is in certain embodiments according to the present invention the conduit section into which the patient's blood that leaves the body of the patient for the purpose of the extracorporeal blood treatment flows and in which it is present before entering the blood treatment device, e.g., a dialyzer.

In certain embodiments according to the present invention, the first section of the arterial conduit section is or encompasses the arterial needle connection to the patient, e.g. the arterial needle connection in a double-needle dialysis method.

The venous conduit section of the extracorporeal blood circuit is in some embodiments of the present invention the conduit section from which the extracorporeally-treated patient's blood flows towards or back into the body of the patient after its treatment in a blood treatment device, e.g., a dialyzer.

In certain embodiments according to the present invention, the first conveying direction—as customary during the blood treatment—corresponds to a conveying direction from an arterial access (blood withdrawal point) of a patient to a blood treatment device, for example a blood filter or a dialyzer and subsequently through the venous line section of the extracorporeal circuit to the venous access (blood return point). All devices which are disposed in the first conveying direction with regard to a reference point of the flow path, are thus positioned downstream. The devices which are disposed with regard to the reference point opposite the first conveying direction, are thus positioned upstream.

The control unit is designed in some particular embodiments according to the present invention as a regulating unit.

The blood treatment apparatus according to the present invention is embodied in some embodiments according to the present invention as hemodialysis apparatus or hemofiltration apparatus or hemodiafiltration apparatus.

The blood treatment apparatus according to the present invention comprises in certain embodiments according to the present invention at least one control unit according to the present invention.

Some or all of the embodiments according to the present invention may exhibit one, several or all of the above and/or below advantages.

An achievable advantage via some of the embodiments of the present invention is that bags can be used as a second source, which exemplarily comprise citrate at a high concentration. The respective used bag lasts therefore for a comparatively long time. Bag change intervals may be accordingly long, which saves time and effort to change the bag, requires only a small storage space for such bags and signifies little effort in procurement and disposal.

Hereby, the content of the second source does not nevertheless tend despite its high concentration to an uneven mixing with the patient's blood, e.g. clot formation; due to the fact that the content is introduced into the patient's blood only after dilution through fresh dialysate or substituate, which takes place in the joint or common conduit.

Another achievable advantage may be that there is no significant fluid balancing error caused by the volume drawn from the second source (e.g. a bag) which is not, as in the examples of the accompanying figures here, usually weighed and therefore is not included in the fluid balancing. Because the volume in the joint or common conduit given to the second fluid may be, due to its high concentration in the second source, comparatively small or very small. It may be very small particularly in comparison with the volume of the first fluid which comes from the first source and is usually subject to weighing as a fresh dialysate or substituate, as seen in the accompanying figures. The major portion or part of the combined fluid—from the first and second fluid —, namely the first fluid, is thoroughly subject to balancing which makes the latter more precise in an advantageous manner.

If the method encompasses detecting a change in the weight of the first source, as provided in some embodiments according to the present invention, then the feed rate of the first conveying device or the volume conveyed by it may be concluded on the basis of the detected weight variations.

This may advantageously serve for monitoring the activity of the first conveying device. An alert or alarm for the user, a regulation or control of the first conveying device and/or otherwise consideration of the information obtained from the scale, which is often highly accurate, anyhow comparatively accurate with regard to the controllability of pumps, that the conveying device does not convey as set. Each result is considered with the known advantages associated thereto.

Hereinafter, the present invention is purely exemplarily described with reference to the accompanying figures. In the figures, same reference numerals refer to the same or identical components or parts. The following applies:

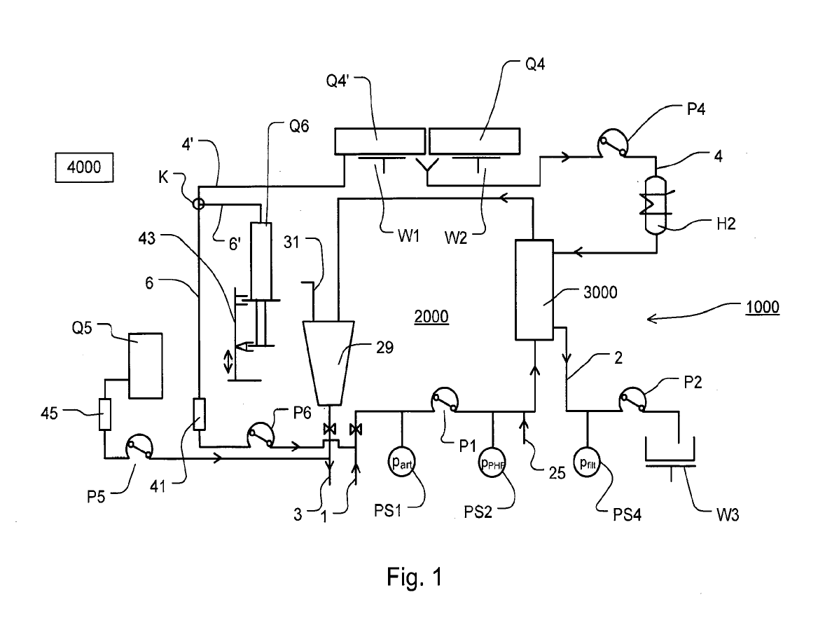

FIG. 1 shows a simplified illustration of a blood treatment apparatus according to the present invention with an extracorporeal blood circuit in a first embodiment; and

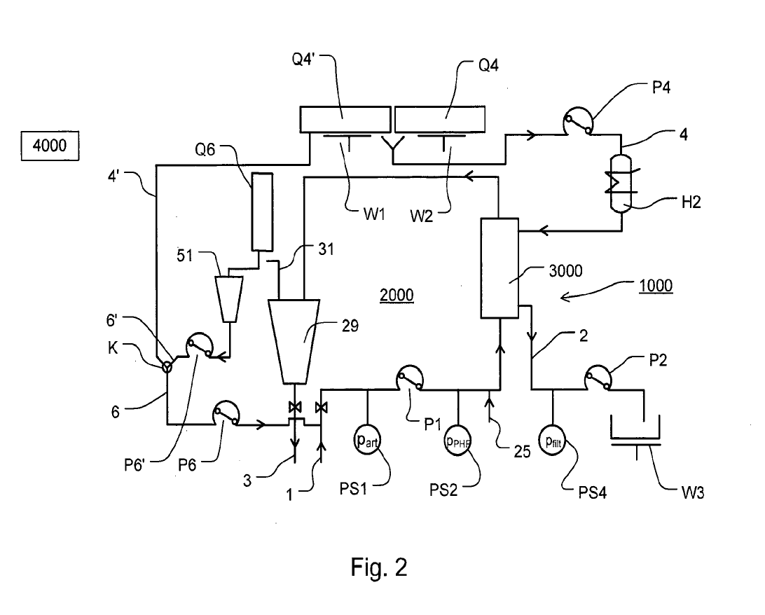

FIG. 2 shows a simplified illustration of a blood treatment apparatus according to the present invention with an extracorporeal blood circuit in a second embodiment;

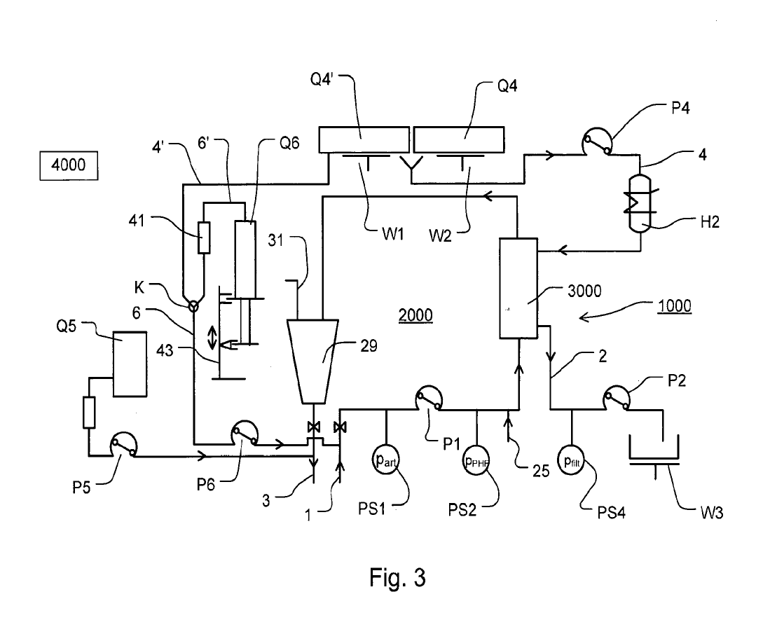

FIG. 3 shows a simplified illustration of a blood treatment apparatus according to the present invention with an extracorporeal blood circuit in a third embodiment;

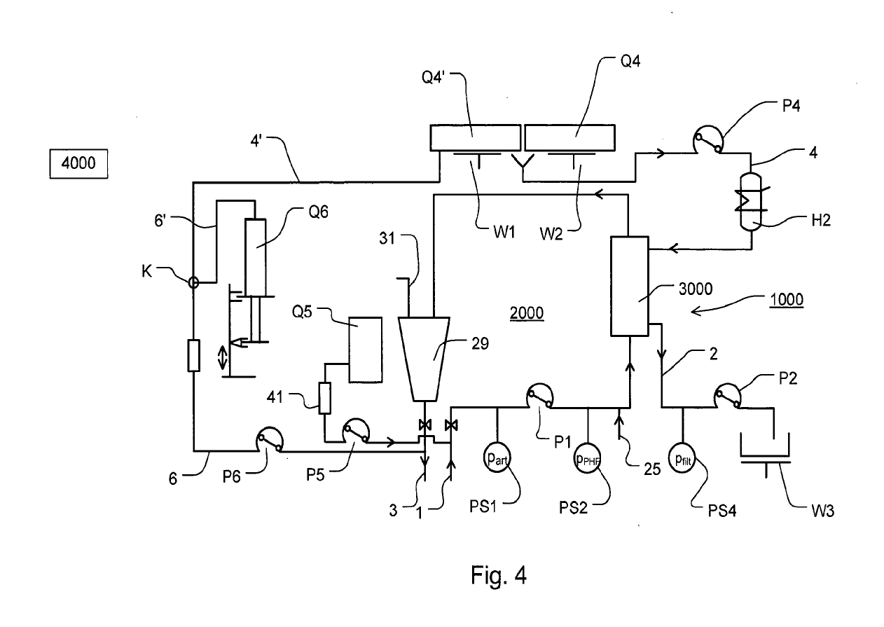

FIG. 4 shows a simplified illustration of a blood treatment apparatus according to the present invention with an extracorporeal blood circuit in a fourth embodiment; and

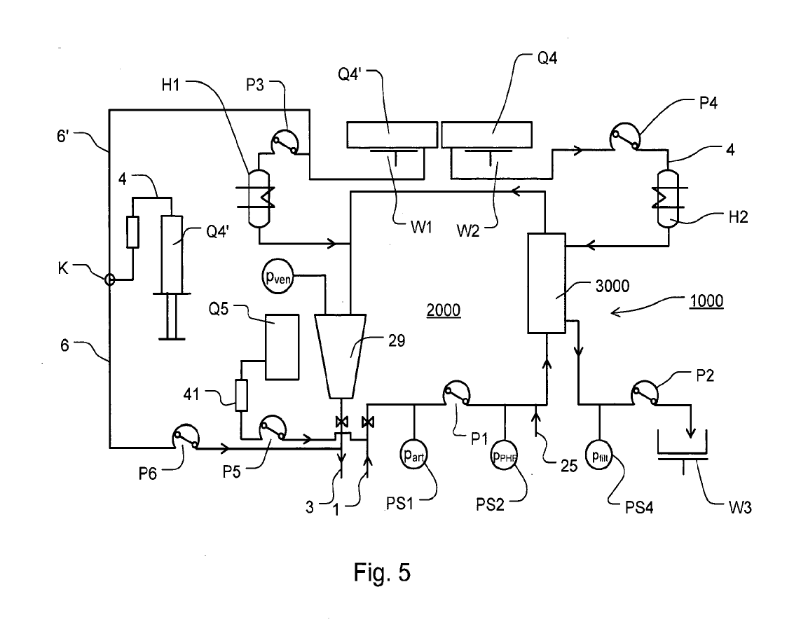

FIG. 5 shows a simplified illustration of a blood treatment apparatus according to the present invention with an extracorporeal blood circuit in a fifth embodiment.

FIG. 1 shows a simplified illustration of a blood treatment apparatus 1000 according to the present invention with an extracorporeal blood circuit 2000.

The extracorporeal blood circuit 2000 comprises or is connected to a blood treatment device 3000, here exemplarily a blood filter or dialyzer. An arterial conduit section 1 of the extracorporeal blood circuit 2000 brings or carries blood from the vascular system of the patient (not shown), towards the blood filter 3000. A venous conduit section 3 of the extracorporeal blood circuit 2000 brings or carries blood from the blood filter 3000 towards the vascular system of the patient (not shown).

The blood treatment apparatus 1000, illustrated in FIG. 1 through only some of its devices, by means of which the method described here is executed, has a blood pump P1. The blood pump P1 conveys blood through portions of the extracorporeal blood circuit 2000 and towards the blood filter 3000, as shown by the small arrowheads generally indicating the flow direction in the figures.

In addition to the aforementioned pump P1, the arrangement shown in FIG. 1 comprises, also purely optional, a number of other pumps P2, P4, P5 and P6, which are provided as purely optional, except for the conveying device P6 which is to be understood in the present embodiment as the first conveying device.

The pump P4 is one of the optional pumps. It is provided to convey/bring fresh dialysate out of a corresponding source Q4, for example a bag, and through an optionally present bag heating with bag H2 using a fresh dialysate conduit 4.

The fresh dialysate supplied in this way exits out of the blood filter 3000 again through a spent dialysate conduit 2, supported by a pump P2, and can be discarded.

The pump P5 is also one of the optional pumps. It supplies or brings a solution of calcium (Ca-solution) out of a source Q5, e.g. a bag, and into the venous conduit section 3.

The pump P6 is not optional, based on the specific embodiment of FIG. 1, wherein pump P6 is to be understood in the example of FIG. 1 as the first conveying device. Pump P6 conveys a combined or mixed fluid along a conduit 6, to be understood here as a joint conduit, into the arterial conduit section 1.

The combined fluid is produced from or at a crossing point K or a connection of a first conduit 4′ with a second conduit 6′ forming the joint first conduit 6 and being a combination or mixture of a first fluid and a second fluid.

The first fluid comes from a first source Q4′, here for example a bag of fresh dialysate (but could also be substituate, saline or another solution or liquid), and is conveyed at first until the crossing point K in the conduit 4′.

The second fluid comes from a second source Q6, here a syringe containing citrate or citrate concentrate (Ci-concentrate), and is conveyed to the crossing point K in a second conduit 6′. The syringe has its own conveying device, a syringe pump which is not shown here. It is evident for the person skilled in the art that such a syringe pump is however not required if the second source Q6 is not a syringe but, for example, a bag. In this case the conveying device is a pump and it is not optional.

The first conduit 4′ and the second conduit 6′ merge or flow from the crossing point or intersection point K into the joint conduit 6.

In the present example, the first source Q4′ and the source Q4 are independent sources for one and the same fluid, namely fresh dialysate. The invention encompasses however also that Q4 and Q4′ are one and the same source or are sources being in fluid communication with each other.

An optional arterial sensor PS1 is provided upstream of the blood pump P1; it measures arterial pressure P_art.

A further, optional pressure sensor PS2 is provided downstream of the blood pump P1, but upstream of the blood filter 3000, and, if provided, of an addition site 25 for heparin. It measures the pressure upstream of the blood filter 3000 (PHF stands for “pre-hemofilter”).

A still further pressure sensor PS4 downstream of the blood filter 3000, but upstream of the pump P2 in the spent dialysate conduit 2, may be provided for measuring a pressure filter of the blood filter 3000.

Blood exiting the blood filter 3000, flows through a venous blood chamber 29 which may comprise a de-aeration device 31.

In the example of FIG. 1, the sources Q4 and Q4′ and the collected or discarded spent dialysate are optionally subject to a balancing. Three scales W1, W2 and W3 are provided for the purpose of the balancing.

The examples shown here corresponds to a gravimetric balancing. The present invention encompasses however also any other balancing, for example by means of balancing chambers.

For example, the crossing point K may be in fluid communication with a balancing unit. The volume proportion of the combined fluid, which does not originate from the second source Q6, i.e. the first fluid, may optionally be compensated for by the balancing unit.

The exemplary arrangement shown in FIG. 1 comprises a first monitoring device 41. It is designed for monitoring the correct function and/or for detecting a flow rate or a flow volume of the first conveying device, in this case the pump P6.

The exemplary arrangement shown in FIG. 1 further comprises a second monitoring device 43. It is designed for monitoring the correct function and/or for detecting a feed rate or a flow volume of the second conveying device, in this case of the syringe pump with the second source Q6. The second monitoring device 43 is, purely optionally, recognizably designed as a distance sensor. Both the first and the second monitoring devices 41, 43 can be designed as a drop counter. Both the first and the second monitoring devices 41, 43 may be connected to a comparing device, not shown in the figures.

Other monitoring devices can also be provided as shown in FIG. 1 in the example of the monitoring device 45.

A control or regulating unit 4000 according to the present invention is indicated in FIG. 1. It is in signal communication with all relevant devices, in any case or in particular with the pump P6.

FIG. 2 shows a simplified illustration of a blood treatment apparatus 1000 according to the present invention with an extracorporeal blood circuit in a second embodiment.

Pump P6′ represents or is the second conveying device. Pump P6′ is located downstream of the second source Q6 and upstream of the crossing point K.

A drip chamber 51 is provided between the second source Q6 and the pump P6′. The drip chamber 51 can be used to monitor via a monitoring device. The embodiment of FIG. 2, comprises in the area of the first and second sources Q4′ and Q6 as well as in the area of the conduit 6, purely exemplarily, only one monitoring device.

The second source Q6 contains merely exemplarily any concentrate.

FIG. 3 shows a simplified illustration of a blood treatment apparatus 1000 according to the present invention with an extracorporeal blood circuit in a third embodiment.

The embodiment of FIG. 3 differs from that of FIG. 1 by the fact that both the first monitoring device 41 and the second monitoring device 43 are in the conduit 6′. The first conduit 6, however, does not have, purely optionally, any monitoring device anymore.

FIG. 4 shows a simplified illustration of a blood treatment apparatus 1000 according to the present invention with an extracorporeal blood circuit in a fourth embodiment.

The embodiment of FIG. 4 differs from that of FIG. 1 by the fact that the pump P6, as a first conveying device, introduces the combined fluid not in the arterial conduit section 1 but rather in the venous conduit section 3.

In addition, reference is made to the elaborations on FIG. 1.

FIG. 5 shows a simplified illustration of a blood treatment apparatus according to the present invention with an extracorporeal blood circuit 2000 in a fifth embodiment.

In the embodiment of FIG. 5, the first source Q4′ comprises a substituate, while the source Q4 contains fresh dialysate. The source Q4′ contains, purely exemplary, calcium concentrate.

The arrangement comprises in contrast to the embodiments of the preceding figures, a further bag heating H1 with a bag for substituate. In order to convey the substituate, a pump P3 is provided downstream of the source Q4′ but upstream of an addition site (may be a valve or may have a valve) for the substituate in post-dilution.

The joint conduit 6 flows into the venous conduit section 3.

The following features, although not shown in the figures, may in each embodiment according to present invention be again purely optional and provided in any combination.

The arterial conduit section 1 may comprise an arterial clamp.

The arterial conduit section 1 may comprise an arterial septum, optionally as an addition device.

The venous conduit section 3 may comprise a venous air bubble detector/optical sensor.

The venous conduit section 3 may comprise a venous clamp.

The blood cassette may comprise a check valve

For the purpose of adding heparin into the interior of the conduit of the extracorporeal blood circuit 2000 or of the blood cassette during an extracorporeal blood treatment, the extracorporeal blood circuit 2000 or the blood cassette may get connected or be connected by means of a corresponding port with an addition site for heparin 25, such as a heparin solution or syringe.

The arterial conduit section 3 may have an arterial bubble detector/optical sensor.

Reference Numeral List

| | |
|---|---|
| 1000 | blood treatment apparatus |
| 2000 | extracorporeal blood circuit |
| 3000 | blood treatment device, blood filter |
| | or dialyzer |
| 4000 | control or regulating unit |
| 1 | arterial conduit section |
| 2 | spent dialysate conduit |
| 3 | venous conduit section |
| 4 | fresh dialysate conduit |
| 4′ | first conduit coming from first |
| | source |
| 6 | joint or common conduit |
| 6′ | second conduit coming from second |
| | source |
| H1 | bag heater with bag |
| H2 | bag heater with bag |
| P1 | blood pump |
| P2, P3, P4, P5 | optional pumps |
| P6 | pump, first conveying device |
| P6′ | pump, second conveying device |
| PS1 | arterial sensor, measures the |
| | arterial pressure P_art |
| PS2 | optional pressure sensor |
| PS4 | pressure sensor for measuring a |
| | filter pressure |
| Q4 | source with fresh dialysate |
| Q4′ | first source |
| Q5 | source |
| Q6 | second source |
| 25 | heparin addition site |
| 29 | venous blood chamber |
| 31 | de-aeration device |
| K | crossing point or intersection point |
| | or connection point of the first |
| | conduit with the second conduit |
| W1, W2, W3 | scales |
| 41 | first monitoring device |
| 43 | second monitoring device |
| 45 | monitoring device |
| 51 | drip chamber |

1.-22. (canceled)

23. A method for providing a solution for a blood treatment of a patient, the method comprising:
providing a blood treatment apparatus comprising: at least one first source of saline solution, fresh dialysate or substituate as a first fluid;
at least one first conduit portion, which connects to the first source;
at least one second source of a second fluid in the form of a concentrate or a solution;
at least one second conduit portion, which connects to the second source;
at least one third conduit portion, which is in fluid communication with a section of an extracorporeal blood circuit and to which both the first conduit portion and the second conduit portion are fluidly connected such that both the first source and the second source are in fluid communication with the third conduit portion; and
a first fluid conveying device arranged to convey a fluid present inside the third conduit portion;
wherein the method comprises:
operating at least the first conveying device such that a combination of the first fluid and the second fluid is conveyed through the third conduit portion.

24. The method of claim 23 wherein the extracorporeal blood circuit comprises a blood cassette.

25. The method according to claim 23, wherein the blood treatment apparatus comprises a second conveying device, and wherein the method comprises: conveying, with the second conveying device, the second fluid out of the second source and into the second conduit portion.

26. The method according to claim 23, wherein the blood treatment apparatus comprises a second conveying device, and wherein the method comprises:
conveying, with the second conveying device, the second fluid out of the second source and into the third conduit portion.

27. The method according to claim 23 further comprising:
conveying the first fluid from the first source into the first conduit portion and conveying the second fluid from the second source into the second conduit portion in a volume relation or a flow relation determined by the user.

28. The method according to claim 23 comprising:
monitoring, with at least one monitoring device, a volume conveyed into the third conduit portion or a respective feed rate into the third conduit portion of at least one of the first conveying device and the second conveying device.

29. The method according to claim 28 further comprising:
detecting, with the at least one monitoring device, a distance, by which a section of the second conveying device is moved; and
comparing, with a comparing device, the detected distance with reference data.

30. The method according to claim 28 further comprising:
counting, with a drop counter, drops of at least one of the first and second fluids; and
comparing, with a comparing device, the detected number of drops with reference data.

31. The method according to claim 23 further comprising:
detecting, with at least one scale, a change of a weight of the first source; and
monitoring a conveying rate of the first conveying device based on the detected change of the weight of the first source.

32. The method according to claim 23, further comprising:
providing the second source as a source of a fluid selected from the group consisting of citrate, calcium solution, and heparin solution.

33. The method according to claim 23,
wherein the blood treatment apparatus is a hemodialysis, hemofiltration, or hemodiafiltration apparatus.

34. A blood treatment apparatus comprising: at least one first source of saline solution, fresh dialysate, or substituate as a first fluid;
at least one first conduit portion, which connects to the first source;
at least one second source of a second fluid in the form of a concentrate or a solution;
at least one second conduit portion, which connects to the second source;
at least one third conduit portion, which is in fluid communication with a section of an extracorporeal blood circuit, and to which both the first conduit portion and the second conduit portion are fluidly connected such that both the first source and the second source are in fluid communication with the third conduit portion; and
a first fluid conveying device arranged to convey a fluid present inside the third conduit portion.

35. The method of claim 34 wherein the extracorporeal blood circuit comprises a blood cassette.

36. The blood treatment apparatus according to claim 34, further comprising a second conveying device, which is arranged to convey the second fluid into the second conduit and/or into the joint or common conduit.

37. The blood treatment apparatus according to claim 34, further comprising a first monitoring device for monitoring the pumping function of the first conveying device.

38. The blood treatment apparatus of claim 37 wherein the first monitoring device is configured to monitor the function of the first conveying device by counting drops of the first fluid, and is further configured to compare, by a comparing device, the detected number of drops with reference data.

39. The blood treatment apparatus according to claim 34 comprising a second monitoring device for monitoring the pumping function of the second conveying device.

40. The blood treatment apparatus of claim 39 wherein the second monitoring device is configured to monitor the function of the second conveying device by counting drops of the second fluid, and is further configured to compare, by a comparing device, the detected number of drops with reference data.

41. The blood treatment apparatus according to claim 36, wherein the second conveying device comprises a syringe pump comprising a stamp, and wherein the second monitoring device is configured to detect the distance by which the stamp of the second conveying device is moved, and to compare, with a comparing device, the detected distance with reference data.

42. The blood treatment apparatus according to claim 34, wherein the second source is a source of a fluid selected from the group consisting of citrate, calcium solution, and heparin solution.

43. The blood treatment apparatus according claim 34, wherein the blood treatment apparatus is configured as a hemodialysis, a hemofiltration, or a hemodiafiltration apparatus.

44. The blood treatment apparatus of claim 34 further comprising a control unit configured to send a control signal to operate at least the first conveying device such that a combination of the first fluid and the second fluid is conveyed through the third conduit portion.

45. A control unit of a blood treatment apparatus, the blood treatment apparatus comprising:
at least one first source of saline solution, fresh dialysate or substituate as a first fluid;
at least one first conduit portion, which connects to the first source;
at least one second source of a second fluid in the form of a concentrate or a solution;
at least one second conduit portion, which connects to the second source;
at least one third conduit portion, which is in fluid communication with a section of an extracorporeal blood circuit and to which both the first conduit portion and the second conduit portion are fluidly connected such that both the first source and the second source are in fluid communication with the third conduit portion;
a first fluid conveying device arranged to convey a fluid present inside the third conduit portion,
wherein the control unit comprises an interface for communicating with a computer program product and wherein the control unit is configured to operate at least the first conveying device such that a combination of the first fluid and the second fluid is conveyed through the third conduit portion.

46. The control unit of claim 44 wherein the computer program product comprises a machine readable medium.

47. The control unit of claim 45 wherein the machine readable medium comprises electrically readable control signals and is selected from the group consisting of a disk, a CD, a DVD, and an EPROM.

48. One or more computer readable media storing instructions that are executable by a processing device, and upon such execution cause the processing device to perform operations comprising:
prompting the mechanical operation of at least a first conveying device of a blood treatment apparatus such that a combination of a first fluid and a second fluid is conveyed through a third conduit portion of the blood treatment apparatus.

49. The computer readable media storing instructions of claim 48 wherein the computer readable media storing instructions are electrically readable control signals and are stored on a device selected from the group consisting of a disk, a CD, a DVD, and an EPROM.
